(19) United States
(12) Patent Application Publication
(10) Pub. No.: US 2011/0004031 A1
(43) Pub. Date: Jan. 6, 2011

(54) GLYCERIN PURIFICATION

(75) Inventors: Rico O. Cruz, Craigmont, ID (US); Maryana Z. Pavlova, Saint Petersburg, FL (US); Nikolay I. Zamfirov, Saint Petersburg, FL (US); Dimitar Y. Zamfirov, Sofia (BG)

Correspondence Address: Schwabe Williamson & Wyatt;PACWEST CENTER, SUITE 1900, 1211 SW FIFTH AVENUE, PORTLAND, OR, 97204, US

(73) Assignee: S.I.I., LLC, Pendleton, OR (US)

(21) Appl. No.: 12/774,639

(22) Filed: May 5, 2010

Related U.S. Application Data
(60) Provisional application No. 61/176,081, filed on May 6, 2009.

(51) Int. Cl. C07C 29/80; C07C 29/76; B01D 3/00; B01D 15/04; B01D 36/00

(52) U.S. Cl. 568/870; 568/869; 202/176

(57) ABSTRACT

Techniques are generally described herein for the purification of glycerin. Embodiments include, but are not limited to, methods, systems, and articles of manufacture. Other embodiments may also be disclosed and claimed. Some techniques described herein include adding methyl alcohol and acid to crude glycerin to form a solution, filtering the solution to remove at least one salt of the acid from the solution, separating the solution into a first layer of free fatty acids and a second layer of at least partially purified glycerin, and distilling off the methyl alcohol from second layer of the solution. The second layer may then undergo one or more additional operations including neutralizing, further filtering to remove an excess of the neutralizing agent, passing through a plurality of ion-exchange columns, deodorizing, and dewatering.

### CROSS-REFERENCE TO RELATED APPLICATION

The present application claims priority to U.S. Provisional Patent Application No. 61/176,081, filed May 6, 2009, entitled “GLYCERIN PURIFICATION,” the entire disclosure of which is hereby incorporated by reference in its entirety for all purposes except for those sections, if any, that are inconsistent with this specification.

### BACKGROUND

Glycerin is produced as a byproduct of a transesterification reaction conducted during production of biodiesel. This glycerin byproduct is in a crude form and is mixed with varying amounts of soap, alcohol, catalyst, and water.

Most of the glycerin can be recovered from the byproduct by using a technique called acidulation. This process uses phosphoric acid to separate the soap into free fatty acids (FFAs) and salts. This method results in separation of the byproduct into three distinct layers: an upper layer of FFAs; a middle layer of salts, and a bottom layer of glycerin. This glycerin will be heavily acidified by this process and will also contain colorants, water, and alcohol (unless the alcohol was removed prior to this process). The glycerin can be distilled to 100% pure, but this is a very costly and difficult process.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 is a flow diagram illustrating some of the operations associated with an example method for purifying glycerin;

FIG. 2 is a flow diagram illustrating some of the operations associated with another example method for purifying glycerin;

FIG. 3 is a block diagram of an example system for purifying glycerin;

FIG. 4 is a schematic illustrating an example system and method for purifying glycerin including at least some operations from the example methods of FIG. 1 and FIG. 2; and

FIG. 5 is a block diagram of an example computer program product for facilitating the purification of glycerin;

all arranged in accordance with various embodiments of the present disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to purifying glycerin while avoiding at least some of the costs and difficulties commonly associated with conventional distillation-based glycerin purification techniques. In various embodiments, glycerin may be purified using a relatively low heat, and may produce ash-free glycerol, with minimum waste. For example, free fatty acid by-products may be used for biofuel, potassium phosphate and potassium sulfate may be used as fertilizer, and calcium soap may be used as a soil amendment. Embodiments include, but are not limited to, methods, systems, and articles of manufacture. Other embodiments may also be disclosed and claimed.

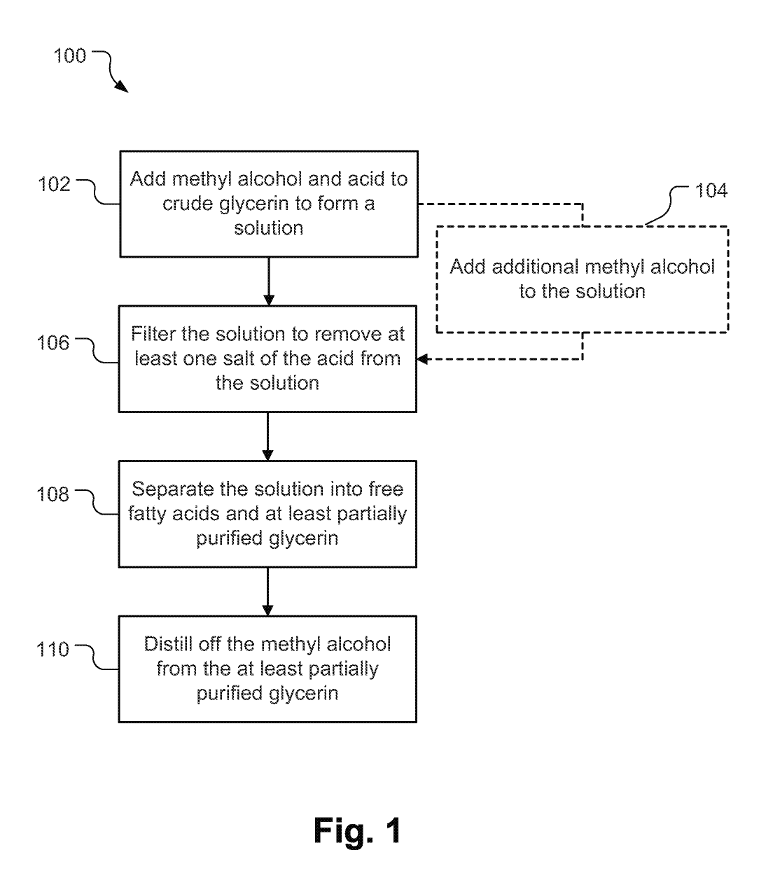

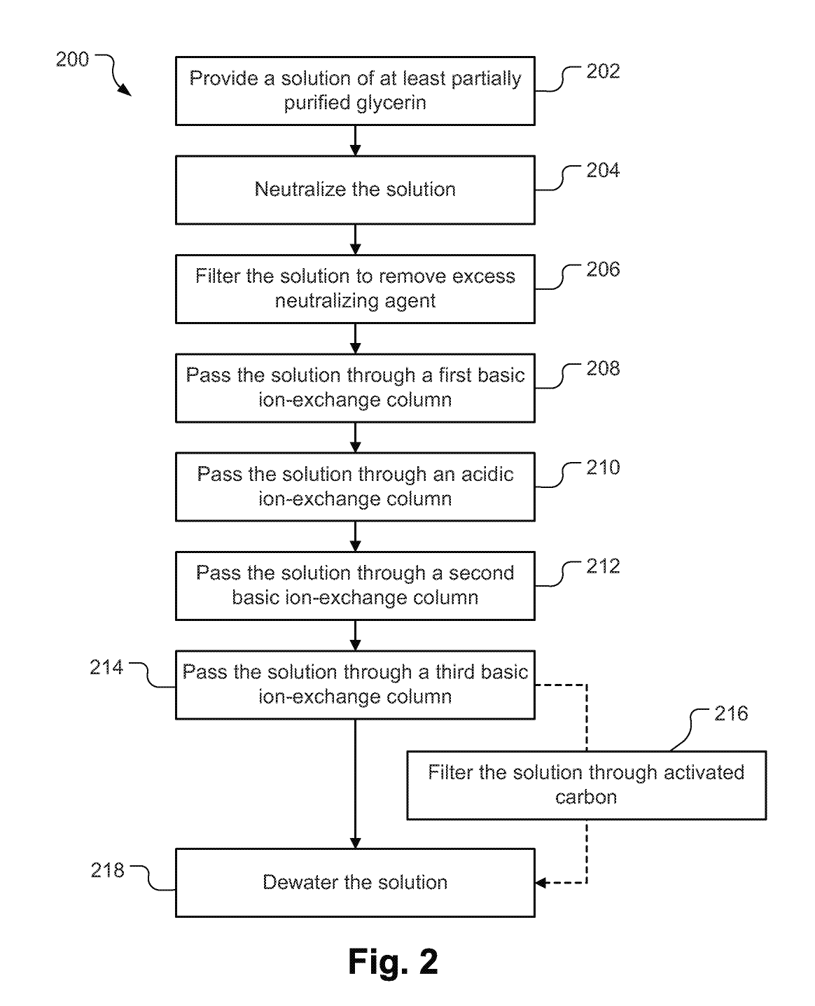

FIG. 1 and FIG. 2 are flow diagrams illustrating some of the operations associated with example methods 100 and 200, respectively, for purifying glycerin, arranged in accordance with at least some embodiments of the present disclosure. The methods 100 and 200 may include one or more functions, operations, or actions of the illustrated blocks. It should be noted that although the methods are illustrated as series of sequential steps, the methods are not necessarily order dependent. Moreover, methods within the scope of this disclosure may include more or fewer steps than that illustrated.

Turning now to FIG. 1, processing for the method 100 may start with block 102 (“Add methyl alcohol and acid to crude glycerin to form a solution”). The crude glycerin may be a byproduct of a transesterification reaction conducted during the production of biodiesel, or may be provided by another source. Although the purity of the crude glycerin may vary depending at least on the source of the crude glycerin, in various embodiments the crude glycerin may have a purity in a range of approximately 50% to 80%.

The crude glycerin, methyl alcohol, and acid may be provided in amounts suitable for forming at least some acid salt crystals for removal in a subsequent operation (to be discussed more fully below). In various embodiments, the crude glycerin may be provided in an amount of approximately 100 parts per volume, and the methyl alcohol (CH₃OH) may be provided in an amount of approximately 20 parts per volume. It is noted that although the starting crude glycerin may include a small quantity of methyl alcohol among the impurities (e.g., colorants, water, salts, etc.), the methyl alcohol added to the crude glycerin may be provided in addition to any amounts of methyl alcohol that may already be present in the crude glycerin itself. In other words, the methyl alcohol and crude glycerin may be provided in relative quantities rather than relative quantities of methyl alcohol and pure glycerin.

The acid may be any one or more acids suitable for adjusting the pH level of the solution. In various embodiments, the acid may comprise sulfuric acid (H₂SO₄), a phosphoric acid (H₂PO₄), hydrochloric acid (HCl), or another acid. The acid may be added to the solution to bring the solution a pH of approximately 2.0 or less. In some embodiments, a quantity of approximately 0.12 parts per volume may be suitable for bringing the pH of the solution to 2.0 or less.

During the operation of adding the methyl alcohol and the acid to the crude glycerin, the solution may be mixed. The mixing may be regular, laminar mixing.

In various embodiments, processing for the method 100 may optionally proceed to block 104 (“Add additional methyl alcohol to the solution”). In various embodiments, additional amounts of methyl alcohol may optionally be added to the solution after providing the acid to the solution in order to regulate the precipitation of at least one salt of the acid. Increasing an amount of the methyl alcohol may facilitate separating free fatty acids and glycerides from salts by solubilizing the free fatty acids and glycerides, resulting in the salts precipitating from the solution.

From either block 102 or block 104, the method 100 may proceed to block 106 (“Filter the solution to remove at least one salt of the acid from the solution”). At this operation, the solution of the crude glycerin, methyl alcohol, and acid may be filtered through a gravity-fed filter, removing at least some salts from the solution. The filtering operation at block 106 may be a quiet filter operation with little to no turbulence.

The particular salts removed by the filtration at block 106 may depend at least in part on the particular acid selected for forming the solution. For example, in embodiments in which phosphoric acid is selected as an acid for forming the solution, at least one salt may include potassium phosphate, while in embodiments in which sulfuric acid is selected, at least one salt may include potassium sulfate. The salts filtered from the solution may be reclaimed for other uses including, for example, for use as a fertilizer.

From block 106, the method 100 may proceed to block 108 (“Separate the solution into free fatty acids and at least partially purified glycerin”). At this operation, the solution may separate into a first layer of free fatty acids and a second layer of at least partially purified glycerin. The first layer may be decanted from the solution and may be reclaimed for other uses, including, for example, for use in producing biodiesel through one or more esterification operations. The remaining solution may comprise the at least partially purified glycerin, methyl alcohol, and water, which may comprise approximately half of the solution.

From block 108, the method 100 may proceed to block 110 (“Distill off the methyl alcohol from the solution”). At this operation, at least some of the methyl alcohol may be distilled off of the solution. In various embodiments, the distillation operation may be a membrane distillation or vacuum distillation. The methyl alcohol distilled off of the solution may be reclaimed for other uses.

In various embodiments, operations 108 and 110 may be reversed such that block 106 may proceed first to distilling off the methyl alcohol, and then proceeding to separating the solution into the first layer of free fatty acids and the second layer of at least partially purified glycerin.

The solution of at least partially purified glycerin may be further purified. FIG. 2 is a flow diagram illustrating some of the operations associated with an example method 200 for purifying a solution of at least partially purified glycerin, arranged in accordance with at least some embodiments of the present disclosure.

Processing for the method 200 may start with block 202 (“Provide a solution of at least partially purified glycerin”). The solution of at least partially purified glycerin may be a solution obtained using the method 100 of FIG. 1, or may be a solution obtained by another method.

From block 202, the method 200 may proceed to block 204 (“Neutralize the solution”). At this operation, a neutralizing agent may be added to the solution to bring the pH to approximately 7.0. The neutralizing agent may be any material suitable for adjusting the pH of the solution. In some embodiments, the neutralizing agent may comprise a soap such as, for example, calcium hydroxide (Ca(OH)₂) also known as hydrated lime.

From block 204, the method 200 may proceed to block 206 (“Filter the solution to remove excess neutralizing agent”). At this operation, the neutralized solution may be filtered to filter off any remaining neutralizing agent. When the neutralizing agent used is calcium hydroxide, the precipitate produced is calcium soap (RCOOCa), where R is a fatty acid chain (monoglyceride). In these embodiments, water may be added to the solution with the neutralizing agent, particularly when the neutralizing agent is in solid form, for faster filtering. The excess neutralizing agent filtered from the solution may be reclaimed for other uses including, for example, for use as a soil amendment.

From block 206, the method 200 may proceed with providing the filtered solution to a number of ion-exchange columns. At block 208 (“Pass the solution through a first basic ion-exchange column”), the solution may be passed through a first basic ion-exchange column. The first basic ion-exchange column may comprise an anion bleaching column that is strongly basic and works to remove color from the solution.

From block 208, the method 200 may proceed to block 210 (“Pass the solution through an acidic ion-exchange column”). At this operation, the solution may be passed through an acidic ion-exchange column. The acidic ion-exchange column may be a cation column that is strongly acidic and works to remove sodium from the solution to soften, or de-mineralize, the solution.

From block 210, the method 200 may proceed to block 212 (“Pass the solution through a second basic ion-exchange column”). At this operation, the solution may be passed through a second basic ion-exchange column. The second basic ion-exchange column may be an anion column that is weakly basic and works to further de-mineralize and electroplate the solution, e.g., by removing iron.

From block 212, the method 200 may proceed to block 214 (“Pass the solution through a mixed ion-exchange column”). At this operation, the solution may be passed through a mixed ion-exchange column. The mixed ion-exchange column may be a mixed anion/cation column that is neutral and works to further de-mineralize and electroplate the liquid. In various embodiments, the first basic ion-exchange column may have a pH that is more basic than a pH of the second basic ion-exchange column and a pH of the mixed ion-exchange column.

From block 214, the method 200 may optionally proceed to block 216 (“Filter the solution through activated carbon”). At this operation, the solution may be optionally passed through an activated carbon filter to remove odor, if present.

From block 214 or block 216, the method 200 may proceed to block 218 (“Dewater the solution”). At this operation, substantially all water may be removed from the solution. The water may be distilled from the solution by any suitable dewatering operating, including, for example, a boiling operation, a vacuum distillation operation, using a molecular sieve, etc. The solution may comprise purified glycerin that may, at least in some embodiments, be approximately 99.9% pure.

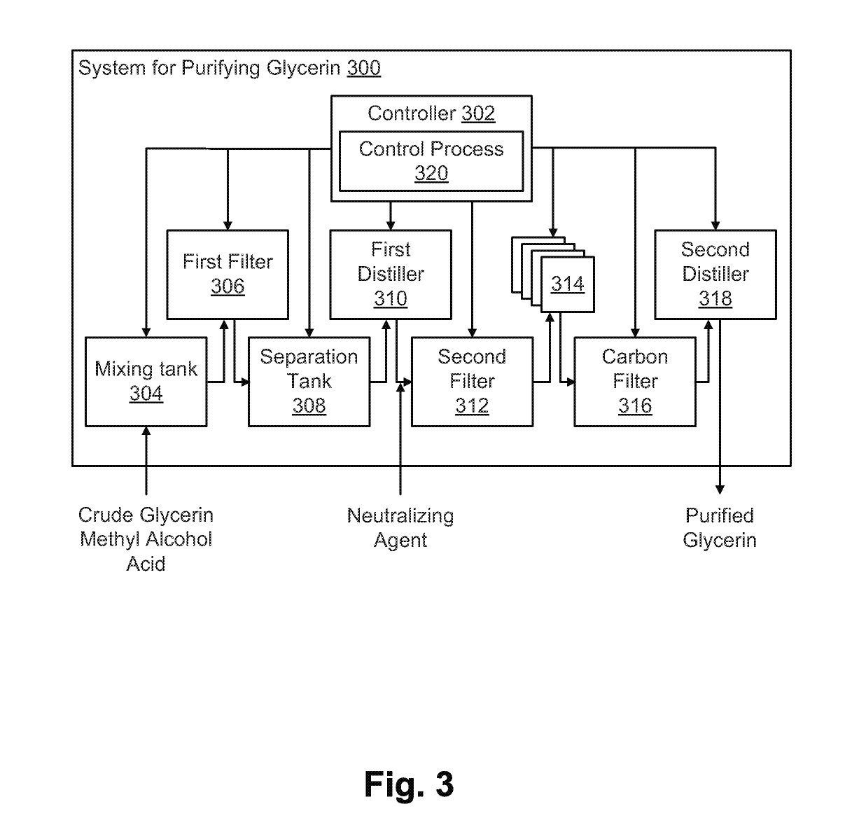

FIG. 3 is a block diagram of an example system for purifying glycerin, arranged in accordance with at least some embodiments of the present disclosure. A basic configuration of the system 300 may include a controller 302, a mixing tank 304, a first filter 306, a separation tank 308, a first distiller 310, a second filter 312, a plurality of ion-exchange columns 314, a carbon filter 316, and a second distiller 318, all coupled together and generally configured as illustrated. It should be noted that systems within the scope of this disclosure may include more or fewer components than that illustrated.

The mixing tank 304 may be a vessel configured to hold a solution of crude glycerin, methyl alcohol, and an acid according to the various methods described herein. The mixing tank 304 may include one or more inlets configured for receiving the crude glycerin, methyl alcohol, and acid, either individually or in one or more pre-mixed combinations. The mixing tank 304 may include a mixer (not illustrated) for providing regular, laminar mixing to the solution. The mixing tank 304 may be heated or un-heated.

The first filter 306 may be configured to receive the solution from the mixing tank 304 and to filter the solution to remove at least one salt of the acid from the solution. The first filter 306 may have a pore size dependent at least in part on the size of the acid salt crystals. In some embodiments, a pore size of approximately 50 microns may be suitable for removing at least some of the acid salt crystals from the solution. The first filter 306 may be a quiet filter for providing filtration with little to no turbulence.

The separation tank 308 may be a vessel configured to receive the solution from the first filter 306 and to separate the solution into a first layer of free fatty acids from the solution and a second layer of at least partially purified glycerin, as described elsewhere herein. The separation tank 308 may include one or more outlets (not illustrated) for decanting and separately routing the free fatty acids and the at least partially purified glycerin.

The first distiller 310 may be configured to receive the solution from the separation tank 308 and to distill off the methyl alcohol from the second layer of the solution. The first distiller 310 may be any suitable distiller including, for example, those allowing for low-temperature distillation. Distillers such as membrane distillers or vacuum distillers may be used in various embodiments.

Although distilling off the methyl alcohol after separating out the free fatty acids may efficiently allow for a lesser volume to be distilled, in various embodiments, the separation tank 308 and the first distiller 310 may be reversed such that a solution may proceed from the first filter 306 to the first distiller 310, and then to the separation tank 308.

The second filter 312 may be configured to receive the solution including the at least partially purified glycerin and to filter the solution to remove excess neutralizing agent from the solution. The second filter 312 may have a pore size dependent at least in part on the size of the neutralizing agent crystals. The second filter 312 may implement a pumped filtering operation.

The plurality of ion-exchange columns 314 may be configured to receive the solution of at least partially purified glycerin and provide ion-exchange filtration of the solution, as described herein. The ion-exchange columns 314 may include resins configured to provide, individually or in combination, removal of at least some color from the solution, at least some sodium from the solution, and at least some iron from the solution. To that end, the ion-exchange columns 314 may include various types of resins. For example, the ion-exchange columns 314 may include a first basic ion-exchange column configured to remove color from the solution, an acidic ion-exchange column configured to remove sodium (de-mineralize) from the solution, a second basic ion-exchange column configured to further de-mineralize and removed ion (electroplate) from the solution, and a mixed ion-exchange column configured to further de-mineralize and electroplate the solution.

The carbon filter 316 may comprise an activated carbon filter configured to remove at least some odor from the solution.

The second distiller 318 may be configured to remove the water from the solution. The second distiller 316 may comprise a simple distillation setup, a vacuum distiller, a flash distiller, a molecular sieve, etc.

The controller 302 may be any device suitable for monitoring, adjusting, and/or controlling a process of purifying glycerin according to the various methods described herein. For example, the controller 302 may be a computing device (e.g., a computer system, a microprocessor, a microcontroller, etc.) or an embedded controller (e.g., an Application Specific Integrated Circuit (ASIC), or some other equivalent). The controller 302 may include a control process 310 that includes one or more instructions for monitoring, adjusting, and/or controlling the process of purifying glycerin according to the various methods described herein. As an example, the control process 310 may include instructions for implementing a method for purifying glycerin, comprising routing the solution of crude glycerin, methyl alcohol, and acid from the mixing tank 304 to the first filter 306, filtering the solution using the first filter 306 to remove at least one salt of the acid from the solution, separating the solution into a first layer of free fatty acids and a second layer of at least partially purified glycerin in the separation tank 308, and distilling off the methyl alcohol from the second layer of the solution using the first distiller 310.

Various instructions processed by the controller 302 may include operating a power source (not illustrated) to control signals (e.g., voltage, current, etc.) for any monitoring, adjusting, and/or controlling the process of purifying glycerin according to the various methods described herein.

It should be noted that systems within the scope of this disclosure may include more or fewer elements than that illustrated. For example, in various embodiments, a system may omit the carbon filter 318, add additional distillers and/or filters, etc.

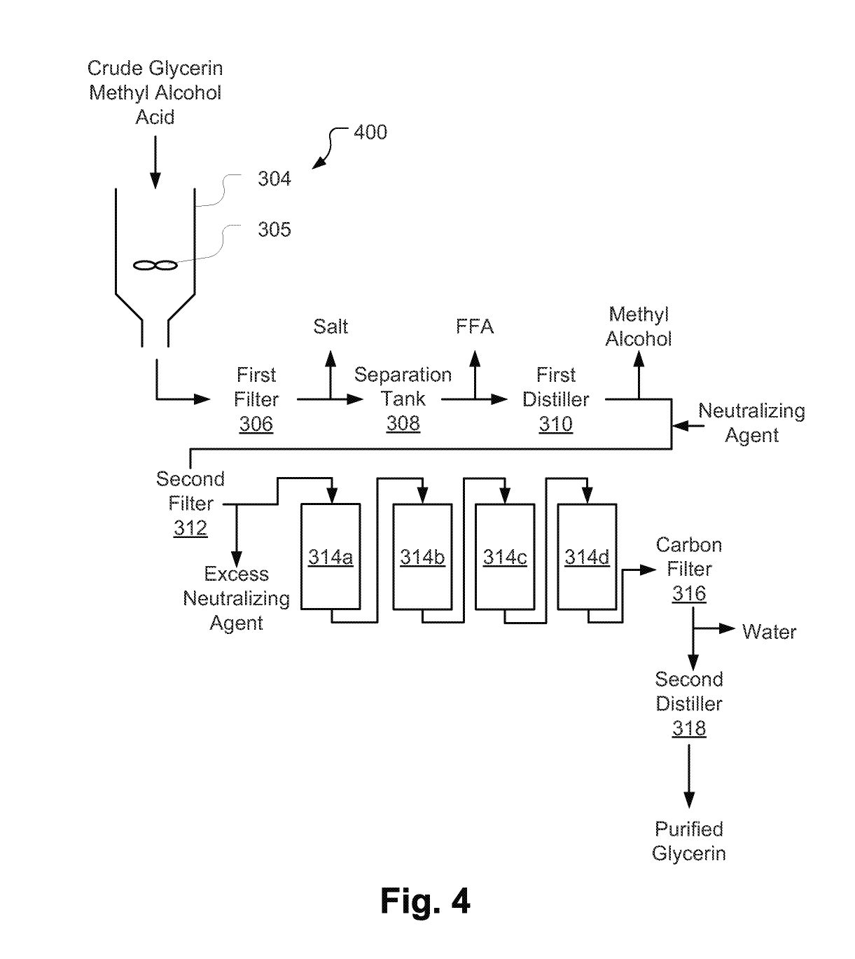

FIG. 4 is a schematic illustrating an example system and method for purifying glycerin, arranged in accordance with at least some embodiments of the present disclosure. The example system includes at least some elements of the system 300 of FIG. 3, and may include at least some operations from the example methods of FIG. 1 and FIG. 2.

At a mixing operation 400, crude glycerin, methyl alcohol, and acid may be provided to the mixing tank 304.

The solution from the mixing operation 400 may then be filtered through the first filter 306 for removing at least some salts of the acid from the solution.

After filtering the solution through the first filter 306, the solution may continue to separation tank 308 and separate into two distinct phases: a first layer of free fatty acids and a second layer of at least partially purified glycerin. The first layer of free fatty acids (FFA) may be decanted from the solution, while the remaining solution of at least partially purified glycerin, methyl alcohol, and water may be further processed.

The second layer of the solution may then undergo a distillation operation using the first distiller 310 for distilling off at least some of the methyl alcohol.

After distilling off the methyl alcohol, a neutralizing agent may be added to the second layer of the solution to bring the pH to approximately 7.0, and any excess neutralizing agent may be filtered using the second filter 312. The filtered solution may then be provided to a number of ion-exchange columns.

The solution may be provided to a first basic ion-exchange column 314a for removing color from the solution. The first basic ion-exchange column 314a may comprise an anion bleaching column that is strongly basic. An example ion-exchange column that may be used as the first basic ion-exchange column 314a is a column that contains Bayer Lewatit S6328A resins.

The solution may then be provided to an acidic ion-exchange column 314b for removing sodium from the solution to soften (de-mineralize) the solution. The acidic ion-exchange column 314b may comprise a cation column that is strongly acidic. An example ion-exchange column that may be used as the acidic ion-exchange column 314b is a column that contains Bayer Lewatit MonoPlus™ S100 resins.

The solution may then be provided to a second basic ion-exchange column 314c for further de-mineralizing and removing iron (electroplating) from the solution. The second basic ion-exchange column 314c may comprise an anion column that is weakly basic. An example ion-exchange column that may be used as the second basic ion-exchange column 314c is a column that contains Bayer Lewatit MonoPlus™ MP64 resins.

The solution may then be provided to a mixed ion-exchange column 314d for further de-mineralizing and electroplating the solution. The mixed ion-exchange column 314d may comprise a mixed anion/cation column that is neutral. An example ion-exchange column that may be used as the mixed ion-exchange column 314d is a column that contains Bayer Lewatit MonoPlus™ MP500 and S100 resins.

After passing through the ion-exchange columns, the solution may be provided to the carbon filter 316 to remove odor from the solution.

The solution may then undergo another distillation operation using the second distiller 318 for dewatering the solution.

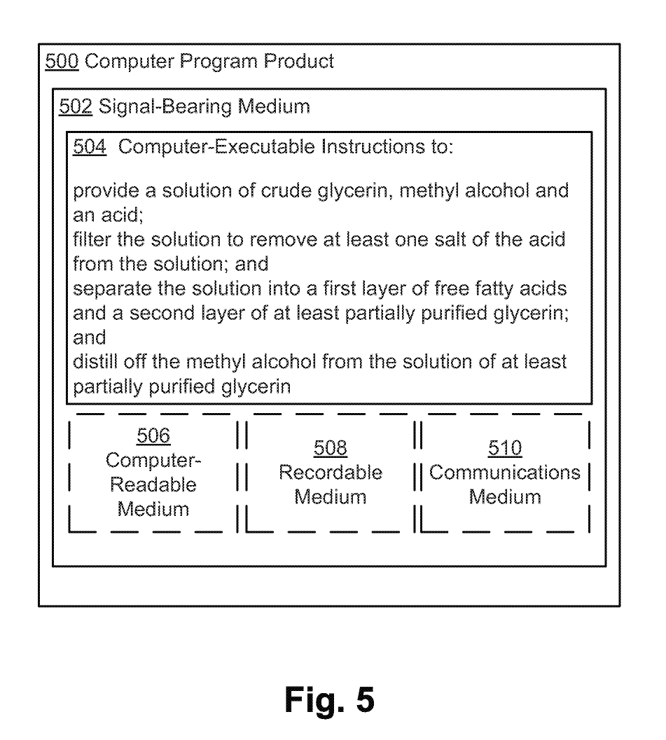

FIG. 5 is a block diagram of an example computer program product for facilitating purification of glycerin, arranged in accordance with at least some embodiments of the present disclosure. In an example, as shown in FIG. 5, the computer program product 500 may include a signal-bearing medium 502 that may include computer-executable instructions 504. The computer-executable instructions 504 may be for providing a solution of crude glycerin, methyl alcohol, and an acid. The computer-executable instructions 504 may also be for filtering the solution to remove at least one salt of the acid from the solution, to separate the solution into a first layer of free fatty acids and a second layer of at least partially purified glycerin, and to distill off the methyl alcohol from the second layer.

Also depicted in FIG. 5, a computer program product 500 may include one or more of a computer-readable medium 506, a recordable medium 508, and a communications medium 510. The dotted boxes around these elements depict different types of mediums included within, but not limited to, a signal-bearing medium 502. These types of mediums may distribute computer-executable instructions 504 to be executed by logic. The computer-readable medium 506 and the recordable medium 508 may include, but are not limited to, a flexible disk, a hard disk drive (HDD), a Compact Disc (CD), a Digital Video Disk (DVD), a digital tape, a computer memory, etc. The communications medium 510 may include, but is not limited to, a digital and/or an analog communication medium (e.g., a fiber-optic cable, a waveguide, a wired communication link, a wireless communication link, etc.).

With respect to the use of substantially any plural and/or singular terms herein, those having skill in the art may translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Various operations may be described as multiple discrete operations in turn, in a manner that may be helpful in understanding embodiments; however, the order of description should not be construed to imply that these operations are order-dependent. Also, embodiments may have fewer operations than described. A description of multiple discrete operations should not be construed to imply that all operations are necessary. Also, embodiments may have fewer operations than described. A description of multiple discrete operations should not be construed to imply that all operations are necessary.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method comprising:
adding methyl alcohol and acid to crude glycerin to form a solution;
filtering the solution to remove at least one salt of the acid from the solution;
separating the solution into a first layer of free fatty acids and a second layer of at least partially purified glycerin; and
after the separating, distilling off the methyl alcohol from the second layer of the solution.

2. The method of claim 1, wherein the crude glycerin has a purity in a range of 50% to 80%.

3. The method of claim 1, wherein the acid comprises sulfuric acid or phosphoric acid.

4. The method of claim 1, wherein the at least one salt comprises potassium phosphate or potassium sulfate.

5. The method of claim 1, wherein the adding comprises mixing together about 100 parts per volume of the crude glycerin, about 20 parts per volume of the methyl alcohol, and about 0.12 parts per volume of the acid.

6. The method of claim 1, wherein the adding comprises adding the acid to the crude glycerin and the methyl alcohol until the solution has a pH of 2.0 or less.

7. The method of claim 1, wherein the filtering comprises gravitationally filtering the solution with substantially no turbulence.

8. The method of claim 1, wherein the distilling comprises distilling off the methyl alcohol from the second layer by membrane distillation or vacuum distillation.

9. The method of claim 1, further comprising:
after the distilling, neutralizing the second layer to a pH of about 7.0;
after the neutralizing, passing the second layer through at least one ion exchange column; and
after the passing the second layer the at least one ion exchange column, dewatering the second layer to remove substantially all water from the second layer.

10. The method of claim 9, further comprising, before the dewatering, filtering the solution through at least one activated carbon filter.

11. A method comprising:
providing a solution including crude glycerin;
separating free fatty acids from the solution;
after the separating, passing the solution through a plurality of ion-exchange columns, the passing including:
passing the solution through a first basic ion-exchange column to remove at least some color from the solution;
passing the solution through an acidic ion-exchange column to remove at least some sodium from the solution;
passing the solution through a second basic ion-exchange column to further remove at least some sodium and at least some iron from the solution; and
passing the solution through a mixed ion-exchange column to further remove at least some sodium and iron from the solution.

12. The method of claim 11, further comprising, prior to the passing the solution through the plurality of ion-exchange columns, neutralizing the solution using a neutralizing agent, and filtering the solution to remove an excess of the neutralizing agent from the solution.

13. The method of claim 12, wherein the neutralizing agent comprises calcium hydroxide.

14. The method of claim 11, wherein the first basic ion-exchange column comprises an ion bleaching column, wherein the acidic ion-exchange column comprises a cation column, wherein the second basic ion-exchange column comprises an anion column, and wherein the mixed ion-exchange column comprises an ion-exchange column including an anion ion-exchange resin and a cation ion-exchange resin.

15. The method of claim 11, wherein the first basic ion-exchange column has a pH that is more basic than a pH of the second basic ion-exchange column and a pH of the mixed ion-exchange column.

16. A system comprising:
a mixing tank configured to hold a solution of crude glycerin, methyl alcohol, and an acid;
a filter configured to receive the solution from the mixing tank and to filter the solution to remove at least one salt of the acid from the solution;
a separation tank configured to receive the solution from the filter and to separate the solution into a first layer of free fatty acids and a second layer of at least partially purified glycerin; and
a distiller configured to receive the second layer of the solution from the separation tank and to distill off the methyl alcohol from the second layer.

17. The system of claim 16, further comprising:
another filter configured to receive the second layer from the distiller to remove an excess of the neutralizing agent from the second layer, and
a plurality of ion-exchange columns configured to receive the second layer from the other filter.

18. The system of claim 17, wherein the ion-exchange columns include:
a first basic ion-exchange column configured to receive the second layer from the other filter;
an acidic ion-exchange column configured to receive the at least partially purified glycerin from the first basic ion-exchange column;
a second basic ion-exchange column configured to receive the at least partially purified glycerin from the acidic basic ion-exchange column; and
a mixed ion-exchange column configured to receive the at least partially purified glycerin from the second basic ion-exchange column.

19. A computer-readable medium having stored thereon, computer-executable instructions that, as a result of execution by a system for purifying glycerin, cause the system to perform a method comprising:
providing a solution of crude glycerin, methyl alcohol and an acid;
filtering the solution to remove at least one salt of the acid from the solution;
separating the solution into a first layer of free fatty acids and a second layer of at least partially purified glycerin; and
after the separating, distilling off the methyl alcohol from the second layer of the solution.

20. The computer-readable medium of claim 19, wherein the instructions, in response to execution by the system, further cause the system to pass the solution, after the separating, using a plurality of ion-exchange columns.
